SEQUENCE LISTING (6 sequences)

SEQ ID NO: 1 - DNA, 606 bp, Homo sapiens
  CDS (17)..(592)
caccagatctcccaccatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaacgaagaaagaacaaaaaacagcccactttcttcctatggcaataactgtcgacggc

SEQ ID NO: 2 - protein, 192 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKRRKNKKQPTFFLWQ

SEQ ID NO: 3 - DNA, 606 bp, Homo sapiens
gccgtcgacagttattgccataggaagaaagtgggctgttttttgttctttcttcgttttttttcctcttacaggaatccccttttgatttaaggcaacaaacatttcccctccgttgtgtgtccatttagctgatgcatatgtgttgtaatggttttcagaattagttctttgaagttacaatcttcattgcattctttctttgcatagagttttccttccttgttcattgcaagatagaattcactttccacccctttgattgccacaattccaactgccactgtcctgatttccatgatattgtaattattcttcatctcttgggtcccttttactttgcctcttttatcgatcctcaggcaccactgtgttcgacagaagagtcttctcactcttatatcccctccttccatgtaatcataacttcttgtgtgtcgctcagggctggaacagttcacatttgtagccatttgctctggagtcatgtcattgcaagctaaagatatagtacccactagacagataatgtgaaagcatgatctgtagagcaaagttggcgggatccatgtcagtatccatttgtgcatggtgggagatctggtg

SEQ ID NO: 4 - protein, 166 aa, Homo sapiens
MHKWILTWIPPTLLYRSCFHIICLVGTISLACNDMTPEQMATNVNCSSPERHTRSYDYMEGGDIRVRRLFCRTQWCLRIDKRGKVKGTQEMKNNYNIMEIRTVAVGIVAIKGVESEFYLAMNKEGKLYAKKECNEDCNFKELILKTITTHMHQLNGHTTEGKCLLP

SEQ ID NO: 5 - DNA, 582 bp, Homo sapiens
atgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataact

SEQ ID NO: 6 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

(19) United States
(12) Patent Application Publication
(10) Pub. No.: US 2005/0074773 A1
(43) Pub. Date: Apr. 7, 2005

(54) Methods for diagnosing and treatment of conditions that alter phosphate transport in mammals

(76) Inventors: Richard A. Shimkets, Guilford, CT (US); Thomas O. Carpenter, Orange, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/644,469

(22) Filed: Aug. 20, 2003

Related U.S. Application Data
(60) Provisional application No. 60/404,659, filed on Aug. 20, 2002.
(60) Provisional application No. 60/463,219, filed on Apr. 16, 2003.

(51) Int. Cl. C12Q 1/68; G01N 33/53

(52) U.S. Cl. 435/6; 435/7.1

(57) ABSTRACT

The present invention describes novel methods for diagnosis and treatment of conditions that alter phosphate transport in mammals. The fibroblast growth factor proteins and nucleotides that may be useful as a therapeutic or in the diagnosis of such conditions are also described.

### RELATED APPLICATIONS

This application claims the benefit of priority from U.S. Provisional Applications U.S. Ser. No. 60/404659, filed 20 Aug. 2002 and U.S. Ser. No. 60/463219, filed 16 Apr. 2003, each of which is incorporated by reference in its entirety.

### FIELD OF THE INVENTION

Phosphorus, is a major component of bone, the most abundant intracellular anion, and among the most abundant tissue constituents. It is critical to and involved in nearly all metabolic processes. The total amount of phosphorus in the normal adult is between 700 and 1,000 grams, of which approximately 85% is found in the skeleton, 15% is found in soft tissues, and 0.1% is found in extracellular fluids. In fasting plasma, most of the phosphorus is present as inorganic orthophosphate in concentrations of 2.8 to 4.0 mg/dL.

In the body, adsorption of phosphorus is under the influence of vitamin D, while phosphorus excretion is under the control of parathyroid hormone (Parathyroid hormone decreases tubular phosphate reabsorption and increases excretion of soluble phosphorus into the urine). The effect of vitamin D on phosphate reabsorption is relatively minor. The concentration of phosphorus in mammals is affected by various factors including age, diet, a large number of pharmaceutical products, and diurnal variations. It is essential for the interpretation of serum levels and urinary clearances that samples be obtained in the fasting state upon which clinical values have been determined and standardized.

Hypophosphatemia has many causes including decreased dietary intake of phosphorus-containing foods, decrease in intestinal absorption, increased excretion into the urine, renal failure, and medications. Unfortunately, the finding of hypophosphatemia is not a reliable indicator of deficiency, since total-body deficiency of phosphorus may be found in a patient's with hyperphosphatemia with, for example, diabetic ketoacidosis.

Hypophosphatemia may be moderate to severe. A common cause of the condition is respiratory alkalosis, and discovery of hypophosphatemia is often the first clinical sign for serious causes of hyperventilation such as sepsis or otherwise unsuspected alcohol withdrawal. Other causes may be phosphorylation of glucose intermediates that may cause cellular uptake of phosphorus with resulting hypophosphatemia. Other common causes of hypophosphatemia include the administration of insulin and consumption of nutrients that stimulate insulin release. Cellular phosphorus uptake also takes place in patients recovering from hypothermia as a result of reactivating metabolism in the patient. Certain malignancies such as fibrosarcomas, prostatic cancers, and possibly small cell cancers of the lung have been reported as additional causes. Still other causes of such hypophosphatemic disorders, specifically hereditary disorders of isolated phosphate wasting, are those such as X-linked hypophosphatemic rickets (XLH), hypophosphatemic bone disease (HBD), hereditary hypophosphatemic rickets with hypercalciuria (HHRH) and autosomal dominant hypophosphatemic rickets (ADHR). ADHR is characterized by low serum phosphorus concentrations, rickets, osteomalacia, lower extremity deformities, short stature, bone pain and dental abscesses.

Severe hypophosphatemia is defined as phosphorus levels in serum below 1.0 mg/dL, and this condition may or may not be accompanied by symptoms such as anorexia, dizziness, bone pain, proximal muscular weakness, and waddling gait. While reduction of serum phosphorus below 1.0 mg/dL suggests severe hypophosphatemia, the condition may not be fully diagnostic. For example, severe hypophosphatemia and severe total body deficiencies of phosphorus may occur in patients with temporary poor dietary intake of phosphorus-containing foods, or in patients consuming phosphate-binding antacids, or with patients presenting early-stage diabetic ketoacidosis.

Hypophosphatemia manifests itself in many different syndromes that may occur simultaneously. In severe hypophosphatemia (which may be aggravated by administration of nutrients to alcoholics or with therapy for diabetic ketoacidosis), elevations in serum creatine phosphokinase (CPK) suggest that the rhabdomyolysis may be superimposed on myopathy. This sequence of events also occurs in experimental phosphate depletion in animals. Severe congestive cardiomyopathy has been noted with chronic hypophosphatemia, and restoration of the phosphorus deficit leads to prompt reversal of the abnormalities. The bone pain and waddling gait seen in hypophosphatemia patients are attributed to the osteomalacia (i.e., failure of normal bone mineralization brought about by the kidney's failure to supply the active form of vitamin D) that develops as a result of phosphate depletion; and the muscular weakness may be due either to direct effects of hypophosphatemia on nerves and muscle or to the effects of hyperparathyroidism that may have a role in the etiology of the hypophosphatemia. Defective growth in children may also be due to phosphate depletion. Hypophosphatemia also results in decreased levels of 2,3-diphosphoglyceric acid and adenosine triphosphate in red blood cells that in turn alter the dissociation of oxyhemoglobin so that less oxygen is delivered in the periphery which mechanism my explain the central nervous system dysfunction seen in hypophosphatemia patients.

Negative phosphorus balance is rarely caused by inadequate phosphorus adsorption in the intestine. Maintenance of normal phosphorus balance is dependent upon efficiency of renal excretion of conservation. In severe renal failure, hyperphosphatemia results from inadequate renal phosphorus clearance; heritable or acquired renal tubular defects may lead to hypophosphatemia due to inadequate renal conservation of phosphorus.

Hyperphosphatemia's defined in adults as an elevation of serum phosphorus above 5 mg/dL. Unfortunately, the condition produces no direct symptoms. However, with maintenance of high phosphorus levels for long periods of time, the driving force for mineralization is increased, and calcium phosphate may be deposited in abnormal sites. Severe hyperphosphatemia is normally associated with extensive cellular or tissue damage. The combination of an increased release of phosphate from damaged muscle tissue and an impaired ability to excrete phosphorous secondary to renal failure (the most common cause of hyperphosphatemia) causes moderate to severe hyperphosphatemia.

Hyperphosphatemia with levels of to 40 mg/dL and above, has occurred secondary to increase absorption from the intestines following administration of excess phosphate salts orally or from the colon as a result of enemas containing phosphate salts. Overmedication with vitamin D, and its production by granulomatous tissue in diseases such as sarcoidosis and tuberculosis may cause hyperphosphatemia. Lactic acidosis is especially important as a cause of hyperphosphatemia. Other causes of hyperphosphatemia may be found in pathologies involving decreased renal excretion such as in the case of renal insufficiency, hypoparathyroidism, hyperostosis, adrenal insufficiency, and infantile hypooshosphatasis; involving intestinal absorption such as in vitamin D ingestion, and granulomatous diseases producing vitamin D (as, for example, tuberculosis); involving internal redistribution of phosphorus such as lactic acidosis, reduced insulin level, acute respiratory acidosis, and lactic acid infusion; involving cellular release of phosphorus such as rhabdomyolysis, tumor lysis, and acute hemolysis; involving parenteral administration of phospholipids infusions or intravenous phosphate salts; and involving spurious hyperphosphatemia such as in thrombocytosis and hyperlipidemia.

Hyperphosphatemia is potentially dangerous because it may lead to hypercalcemia resulting in metastatic calcifications in extraordinary sites such as muscle tissue, or within the cardiovascular system. Known causes of hyperphosphatemia that may lead to hypercalcemia include hypothermia, massive hepatic failure, and hematologic malignancies either because of high cell turnover as part of the malignancy or because of cell destruction when chemotherapy is instituted.

The threat of calcification occurrence from the widespread deposition of calcium disappears after restoration of phosphate levels to normal ranges. At the present time, treatment of hyperphosphatemia involves the use of aluminum-based antacids that bind phosphorus in the lumen of the gastrointestinal tract and prevent its absorption. Although long-term use of aluminum-based antacids should be avoided because they may result in aluminum toxicity, short-term use is acceptable.

The present invention is based upon the new and unexpected finding of a new basis upon which to diagnose and treat hypophosphatemia conditions in mammals, and a new and unexpected treatment for mammalian hyperphosphatemia.

Accordingly, it is one aspect of the present invention to describe a method of diagnosing a hypophosphatemic disorder in a mammal.

It is another aspect of the present invention to describe a biologically-active biopolymer for the treatment of hypophosphatemic disorders in a mammal.

Yet another aspect of the present invention is to describe a biologically-active biopolymer for the treatment of hyperphosphatemic disorders in a mammal.

These and other aspects of the present invention will become more readily apparent to the reader in the following discussion and description, both provided for purposes of disclosure and clarity and not as a limitation to the scope of the invention being described, taken with the accompanying drawings.

### SUMMARY OF THE INVENTION

In one aspect, the invention is drawn to methods of diagnosing a hypophosphatemic condition(s) in a mammal. The methods include the steps of (a) obtaining a biological sample from the mammal and (b) contacting the biological sample with a reagent which detects the presence or absence of a mutation in a nucleic acid encoding FGF7. The presence of the mutation is an indication that the mammal is afflicted with the hypophosphatemic condition(s).

The biological sample can be selected from the group consisting of blood and urine. The reagent can be a nucleic acid. It can also be detectably labeled. In one embodiment, the reagent is detectably labeled with a label selected from the group consisting of a radioisotope, a bioluminescent compound, a chemiluminescent compound, a fluorescent compound, a metal chelate, and an enzyme.

In another aspect, the invention is drawn to methods of diagnosing a hypophosphatemic condition(s) in a mammal. These methods include the steps of (a) obtaining a biological sample from the mammal and (b) contacting the biological sample with a reagent which detects the presence or absence of a mutant form of FGF7 polypeptide, wherein the presence of the mutant form of FGF7 polypeptide is an indication that the mammal is afflicted with the hypophosphatemic condition(s).

The biological sample can be selected from the group consisting of blood and urine. The reagent can be an antibody.

The invention is also drawn to methods of diagnosing a hypophosphatemic condition(s) in a mammal, including the steps of (a) obtaining a biological sample from the mammal and (b) contacting the biological sample with a reagent that detects the level of FGF7 polypeptide in the sample. Wherein an elevated level of FGF7 polypeptide in the sample, relative to the level of FGF7 polypeptide in a sample obtained from a control mammal, is an indication that the mammal is afflicted with the hypophosphatemic condition(s).

The biological sample can be selected from the group consisting of blood and urine. The reagent can be an FGF7 antibody. The reagent can also be detectably labeled. In one embodiment, the reagent is detectably labeled with a label selected from the group consisting of a radioisotope, a bioluminescent compound, a chemiluminescent compound, a fluorescent compound, a metal chelate, and an enzyme.

Moreover, the invention is also drawn to methods of diagnosing osteomalacia in a patient. The methods include the steps of (a) obtaining a biological sample from the patient and (b) detecting the expression or lack thereof of FGF7 in the sample, wherein the expression of FGF7 is indicative of osteomalacia.

The invention is drawn to methods of treating a hypophosphatemic condition(s) in a mammal by administering to a mammal afflicted with the disorder a therapeutically effective amount of a FGF7 inhibitor selected from the group consisting of an inhibitor which reduces the level of mRNA encoding FGF7 polypeptide in the mammal, an inhibitor which reduces the level of FGF7 polypeptide in the mammal, and an inhibitor of the biological activity of FGF7 in the mammal. The inhibitor can be selected from the group consisting of an antisense nucleic acid, a ribozyme, an antibody, a small molecule, a peptide, and a peptidomimetic.

In addition, the invention is also drawn to methods of treating a hyperphosphatemic condition(s) in a mammal. The methods include administering to a mammal afflicted with the disorder a therapeutically effective amount of an isolated nucleic acid encoding FGF7. The isolated nucleic acid comprises a mutation that confers increased stability to the FGF7 polypeptide encoded thereby.

Likewise, the invention is drawn to methods of treating a hyperphosphatemic condition(s) in a mammal by administering to a mammal afflicted with the disorder a therapeutically effective amount of an isolated FGF7 polypeptide. The FGF7 polypeptide can have a mutation that confers increased stability to the FGF7 polypeptide.

Furthermore, the invention is also drawn to methods of treating a hyperphosphatemic condition(s) in a mammal. These methods include administering to the mammal afflicted with, a therapeutically effective amount of a reagent that increases the level of FGF7 polypeptide in the mammal. In one embodiment, the reagent can inhibit degradation of the FGF7 polypeptide.

The invention is also drawn to methods of treating a hyperphosphatemic condition(s) in a mammal, by administering to a mammal afflicted with, a therapeutically effective amount of a population of cells comprising an isolated nucleic acid encoding FGF7. The isolated nucleic acid can have a mutation that confers increased stability on the FGF7 encoded thereby.

The invention is additionally drawn to methods of treating a condition involving deposition of calcium and phosphate in the arteries or soft tissues of a mammal. The methods include administering to the mammal a therapeutically effective amount of FGF7 or a reagent that increases the level of FGF7 polypeptide.

### BRIEF DESCRIPTION OF THE DRAWINGS

FIG. 1: An example trace set of the cell lines with positive (top) and negative(bottom) activity are shown below. The gene corresponding to the peak at position 354.5 encodes the protein known as osteoprotegerin. In the absence of a competing oligonucleotide (a.k.a “poisoning”) the peak is abundant at position 354.5 (red trace) but in the presence of an unlabelled, competing oligonucleotide specifically designed against the known sequence of the osteoprotegerin transcript, the peak is completely diminished (green trace).

FIG. 2: Summary of Gene Expression results showing the identities of the 12 bands identified in the phosphate wasting positive cells.

FIG. 3: Phosphate transport in renal epithelial cells in the presence of various concentrations of recombinant FGF7.

FIG. 4: Phosphate transport in renal epithelial cells in the presence of FGF7 antibody. acm: active condition media; acm+fgfab: active condition media+anti-FGF7 antibody (10 μg/ml).

### DETAILED DESCRIPTION OF THE INVENTION

Phosphate is a nutrient essential for many biological processes including skeletal mineralization and energy metabolism. The homeostasis of the plasma phosphate level is essential for these processes. The reabsorption of phosphate in the kidney is a major determinant of the plasma phosphate level in humans. This phosphate transport occurs primarily via the NaPi2 class of cotransporters in the proximal renal tubule. One such acquired disorder of phosphate homeostasis is oncogenic osteomalacia which is also referred to as tumor-induced osteomalacia (TIO). TIO is marked by renal phosphate-wasting disorder resulting in low serum phosphorus concentration and osteomalacia. Removal of the tumor normalizes phosphate metabolism. Additionally recent studies have identified that phosphatonin to be identical to fibroblast growth factor 23 (FGF-23) (Shimada et al, Proc. Natl. Acad. Sci., 2001, vol:98, 6500-6505). FGF-23 is the recently identified member of the FGF family. While previous studies suggest that overproduction of FGF23 causes TIO, there is speculation that mutation in FGF-23 gene results in autosomal dominant hypophosphatemic rickets (ADHR) (White et al, Nat. Genet. 2000, 26:345-348). ADHR is yet another phosphate wasting disorder resulting in low serum phosphorus concentration, rickets and osteomalacia. Previous studies show no evidence that recombinant FGF-23 can inhibit phosphate uptake in renal proximal epithelial cells.

The present invention differs from the existing literature in that the fibroblast growth factor 7 (FGF7) inhibited phosphate levels in the tumor cell cultures explanted form TIO patients.

Medium from cultures of tumor cells derived from 2 individuals affected with TIO inhibited Pi uptake in vitro. RNA from cultures expressing inhibitory activity were compared with RNA from tumor-derived cultures in which inhibitory activity was not elicited, and candidate proteins specifically expressed by inhibitory cultures were identified. One candidate, fibroblast growth factor 7 (FGF7), potently inhibited Pi uptake in vitro. Antibody to FGF7 reversed FGF7-dependent Pi transport inhibition, and reversed inhibitory activity in conditioned medium from tumor cell cultures. Immunoassay revealed markedly high concentrations of FGF7 in inhibitory conditioned medium, whereas control medium had no detectable FGF7.

The present invention describes the novel use of FGF7 and other fibroblast growth factors in inhibition of phosphate transport. The proteins, antibodies to, small molecules to, and inhibitors of FGF7 may have diagnostic and therapeutic use and have important implications in various phosphate wasting disorders as described above.

Methods that may be employed to identify relevant biological macromolecules include any procedures that detect differential expression of nucleic acids encoding proteins and polypeptides associated with the disorder, as well as procedures that detect the respective proteins and polypeptides themselves. Some of the technologies used in the current invention are as follows:

GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Proper serum phosphate concentrations are maintained by a complex and poorly understood process. Identification of genes responsible for inherited disorders involving disturbances in phosphate homeostasis may provide insight into the pathways that regulate phosphate balance. Phosphate levels in clinical pathological procedures are determined in patient's blood or urine samples.

The present invention identifies a set of proteins and polypeptides, including naturally occurring polypeptides, precursor forms or proproteins, or mature forms of the polypeptides or proteins, which are implicated as targets for therapeutic agents in the treatment of various diseases, pathologies, abnormal states and conditions. A target may be employed in any of a variety of screening methodologies in order to identify candidate therapeutic agents which interact with the target and in so doing exert a desired or favorable effect. The candidate therapeutic agent is identified by screening a large collection of substances or compounds in an important embodiment of the invention. Such a collection may comprise a combinatorial library of substances or compounds in which, in at least one subset of substances or compounds, the individual members are related to each other by simple structural variations based on a particular canonical or basic chemical structure. The variations may include, by way of nonlimiting example, changes in length or identity of a basic framework of bonded atoms; changes in number, composition and disposition of ringed structures, bridge structures, alicyclic rings, and aromatic rings; and changes in pendent or substituents atoms or groups that are bonded at particular positions to the basic framework of bonded atoms or to the ringed structures, the bridge structures, the alicyclic structures, or the aromatic structures.

A polypeptide or protein described herein, and that serves as a target in the screening procedure, includes the product of a naturally occurring polypeptide or precursor form or proprotein. The naturally occurring polypeptide, precursor or proprotein includes, e.g., the full-length gene product, encoded by the corresponding gene. The naturally occurring polypeptide also includes the polypeptide, precursor or proprotein encoded by an open reading frame described herein. A “mature” form of a polypeptide or protein arises as a result of one or more naturally occurring processing steps as they may occur within the cell, including a host cell. The processing steps occur as the gene product arises, e.g., via cleavage of the amino-terminal methionine residue encoded by the initiation codon of an open reading frame, or the proteolytic cleavage of a signal peptide or leader sequence. Thus, a mature form arising from a precursor polypeptide or protein that has residues 1 to N, where residue 1 is the N-terminal methionine, would have residues 2 through N remaining. Alternatively, a mature form arising from a precursor polypeptide or protein having residues 1 to N, in which an amino-terminal signal sequence from residue 1 to residue M is cleaved, includes the residues from residue M+1 to residue N remaining. A “mature” form of a polypeptide or protein may also arise from non-proteolytic post-translational modification. Such non-proteolytic processes include, e.g., glycosylation, myristylation or phosphorylation. In general, a mature polypeptide or protein may result from the operation of only one of these processes, or the combination of any of them.

As used herein, “antibodies” encompass antibodies and antibody fragments, such as Fab, (Fab)₂ or single chain FV constructs, that bind immunospecifically to any of the proteins of the invention. Also encompassed within the invention are peptides and polypeptides comprising sequences having high binding affinity for any of the proteins of the invention, including such peptides and polypeptides that are fused to any carrier particle (or biologically expressed on the surface of a carrier) such as a bacteriophage particle.

Probes based on the human FGF nucleotide sequences can be used to detect transcripts or genomic sequences encoding the same or homologous proteins. In various embodiments, the probe further comprises a label group attached thereto, e.g. the label group can be a radioisotope, a fluorescent compound, a bioluminescent compound, a chemiluminescent compound, a metal chelate, an enzyme, or an enzyme co-factor. Such probes can be used as a part of a diagnostic test kit for identifying cells or tissues which mis-express an FGF protein, such as by measuring a level of an FGF-encoding nucleic acid in a sample of cells from a subject e.g., detecting FGF mRNA levels or determining whether a genomic FGF gene has been mutated or deleted.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an FGF protein, mRNA, or genomic DNA in the preadministration sample; (iii) obtaining one or more post-administration samples from the subject; (iv) detecting the level of expression or activity of the FGF protein, mRNA, or genomic DNA in the post-administration samples; (V) comparing the level of expression or activity of the FGF protein, mRNA, or genomic DNA in the pre-administration sample with the FGF protein, mRNA, or genomic DNA in the post administration sample or samples; and (vi) altering the administration of the agent to the subject accordingly. For example, increased administration of the agent may be desirable to increase the expression or activity of FGF to higher levels than detected, i.e., to increase the effectiveness of the agent. Alternatively, decreased administration of the agent may be desirable to decrease expression or activity of FGF to lower levels than detected, i.e., to decrease the effectiveness of the agent.

Another aspect of the invention pertains to methods of modulating FGF expression or activity for therapeutic purposes. The modulatory method of the invention involves contacting a cell with an agent that modulates one or more of the activities of FGF protein activity associated with the cell. An agent that modulates FGF protein activity can be an agent as described herein, such as a nucleic acid or a protein, a naturally-occurring cognate ligand of an FGF protein, a peptide, an FGF peptidomimetic, or other small molecule. In one embodiment, the agent stimulates one or more FGF protein activity. Examples of such stimulatory agents include active FGF protein and a nucleic acid molecule encoding FGF that has been introduced into the cell. In another embodiment, the agent inhibits one or more FGF protein activity. Examples of such inhibitory agents include antisense FGF nucleic acid molecules and anti-FGF antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an FGF protein or nucleic acid molecule. In one embodiment, the method involves administering an agent (e.g., an agent identified by a screening assay described herein), or combination of agents that modulates (e.g., up-regulates or down-regulates) FGF expression or activity. In another embodiment, the method involves administering an FGF protein or nucleic acid molecule as therapy to compensate for reduced or aberrant FGF expression or activity.

The present invention described herein, i.e., methods for diagnosing and treating conditions that alter phosphate transport in mammals is the result of finding and identifying a biologically-active biopolymer that reduces serum phosphate levels in mammals. More specifically, a protein molecule has been identified that shows new and unexpected activity in reducing serum phosphate. This finding suggests that the biopolymer may be useful in the treatment of those disease states wherein serum phosphate levels are high, and it also suggested suggesting that antagonism of this protein, or its pathway, may be useful in the treatment of serum hypophosphatemia conditions under various disease pathologies in which low phosphate levels are found. In very significant embodiments of the present invention, the biological macromolecules implicated in these pathologies and conditions are proteins and polypeptides, and in such cases the present invention is related as well to the nucleic acids that encode them.

The biologically-active biopolymer that has been unexpectedly found to reduce phosphate levels in cultures from TIO patients according to the present invention is a member of the fibroblast growth factor family, specifically FGF 7 (P21781) and its variants. The biopolymer according to the present invention may, for use in the reduction of phosphate levels be isolated and purified from natural sources, or may be in the form of recombinant FGF 7.

Other active polypeptides that are discussed with supportive data in the current disclosure, to be effective in phosphate transport and related conditions discussed above, are identified below. The gene sequences of these are available in the public domain. Accession No. for each of the gene identified below is given in parentheses. Sequences in GenBank may have been modified.
Glia-derived neurite promoting factor, GDNPF (P07093)
Homo sapiens insulin-like growth factor binding protein 5 (L27556)
Homo sapiens Osteoprotegerin ligand (014788)
Homo sapiens Cathepsin B (P07858)
Homo sapiens CD4 (P01730)

### EXAMPLE 1

Two patients enrolled in the Yale Pediatric Endocrine clinic were identified as having tumor induced osteomalacia (TIO). Tumors explanted from these patients were minced and cultured in 3-4 petri dishes per tumor. Confluent cultures of mixed cellularity (predominantly with fibroblastic and osteoblastic features) were achieved. At biweekly intervals, conditioned media from the cultures were tested for their capacity to inhibit phosphate (P) transport in vitro, using a standard renal epithelial cell assay. One culture from each patient consistently demonstrated substantial inhibition of P transport. One to two cultures never expressed activity and one culture had intermediate, transient activity. After 4 weeks, mRNA from each culture was prepared. P transport characteristics of the medium was confirmed. mRNA from cultures producing the greatest activity and those with no activity were used in the differential gene expression profiling method to determine which genes were specifically expressed by cultures eliciting P transport inhibitory activity.

The following sections describe the study design(s) and the techniques used to identify the Phosphate-Altering Protein Set—encoded protein and any variants, thereof, as being suitable as protein therapeutics, diagnostic markers, targets for an antibody therapeutic and targets for hyperphosphatemia.

CA.11 Phosphate Wasting Factor: Patient Sample and Profile

A very rare tumor type (osteomalacia) has been shown in multiple patients to produce a factor which potently causes phosphate excretion. These tumors lose activity after multiple passages in culture, and thus provide an ideal experimental condition for the identification of the phosphate-wasting factor. Cells from 2 patients were obtained which showed positive activity. After passaging, these cells lost activity. The cells with activity were compared to those that lost activity, and genes expressed in the phosphate-wasting positive cells but absent or at less abundance in the phosphate-wasting negative cells were highlighted for further study.

Method of Identifying the Differentially Expressed Gene and Gene Product

The GeneCalling™ method makes a comparison between experimental samples in the amount of each cDNA fragment generated by digestion with a unique pair of restriction endonucleases, after linker-adaptor ligation, PCR amplification and electropherographic separation. Computer analysis is employed to assign potential identity to the gene fragment.

Three methods are routinely used in the identification of a gene fragment found to have altered expression in models of or patients with obesity and/or diabetes.

A) Direct Sequencing

The differentially expressed gene fragment is isolated, cloned into a plasmid, and sequenced. Afterwards, the sequence information is used to design an oligonucleotide corresponding to either or both termini of the gene fragment. This oligonucleotide, when used in a competitive PCR reaction, will ablate the electropherographic band from which the sequence is derived.

B) Competitive PCR

In competitive PCR, the electropherographic peaks corresponding to the gene fragment of the gene of interest are ablated when a gene-specific primer (designed from the sequenced band or available databases) competes with primers in the linker-adaptors during the PCR amplification.

C) PCR with Perfect or Mismatched 3′ Nucleotides (TraPping)

This method utilizes a competitive PCR approach using a degenerate set of primers that extend one or two nucleotides into the gene-specific region of the fragment beyond the flanking restriction sites. As in the competitive PCR approach, primers that lead to the ablation of the electropherographic band add additional sequence information. In conjunction with the size of the gene fragment and the 12 nucleotides of sequence derived from the restriction sites, this additional sequence data can uniquely define the gene after database analysis.

Results

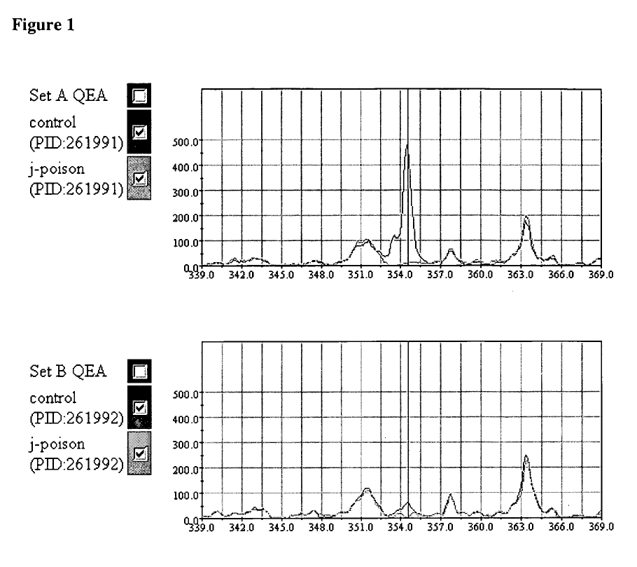

FIG. 1 is an example trace set of the cell lines with positive (top) and negative(bottom) activity are shown below. The gene corresponding to the peak at position 354.5 encodes the protein known as osteoprotegerin. In the absence of a competing oligonucleotide (a.k.a “poisoning”) the peak is abundant at position 354.5 (red trace) but in the presence of an unlabelled, competing oligonucleotide specifically designed against the known sequence of the osteoprotegerin transcript, the peak is completely diminished (green trace).

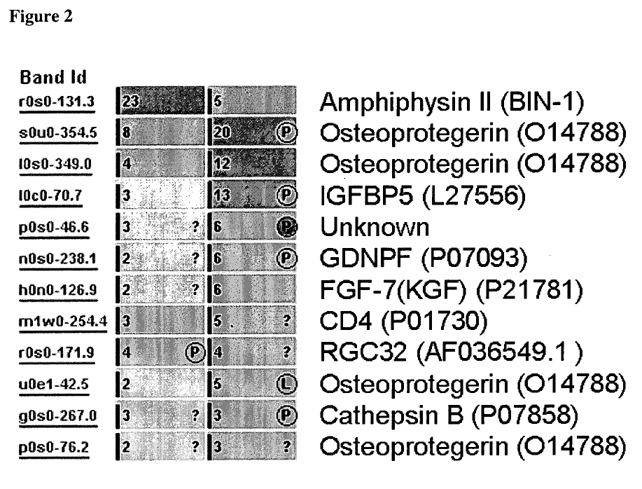

Approximately 4000 gene fragments were analyzed using GeneCalling on the osteomalacia patient samples. Of these, only 12 fragments were consistently upregulated in cells positive for the phosphate transport inhibiting activity which are shown in FIG. 2. Six of the twelve are secreted proteins that are known in the public domain:
Glia-derived neurite promoting factor (GDNPF) (P07093)
Homo sapiens insulin-like growth factor binding protein 5 (L27556)
Homo sapiens Osteoprotegerin ligand (O14788)
Homo sapiens Cathepsin B (P07858)
Homo sapiens FGF-7 (KGF) (P21781)
Homo sapiens CD4 (P01730)

### EXAMPLE 2

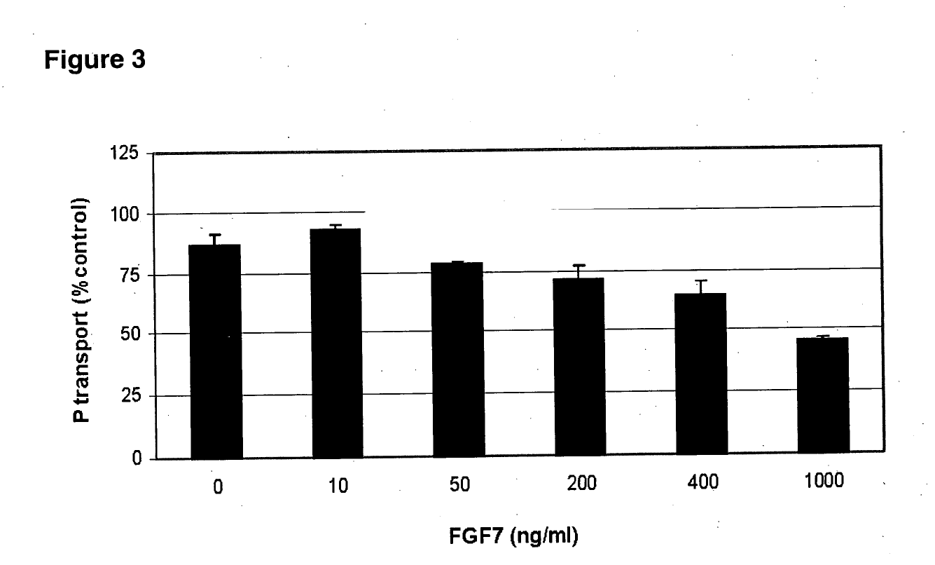

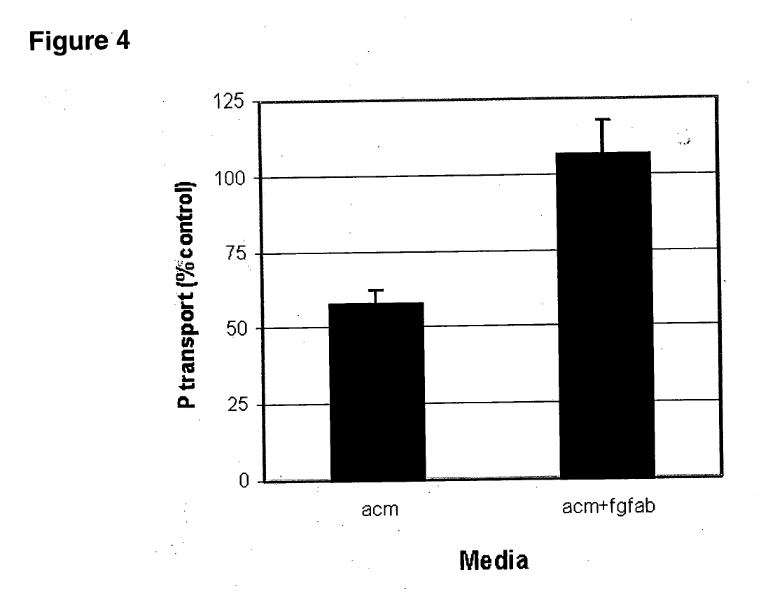

Measurement of phosphate transport was performed in cultured renal proximal tubular epithelial cell line using opossum kidney (OK) cells, in the presence of FGF 7. The phosphate uptake in OK cells was determined according to the standard method known in the art. FIG. 3 shows that FGF 7 can inhibit Phosphate transport in a dose-dependant manner within the physiological range. Furthermore, FIG. 4 demonstrates that FGF-7 antibody can reverse FGF-dependent Phosphate transport inhibition in renal epithelial cells.

Taken together these data suggest that FGF 7 protein can be therapeutically used in hyperphosphatemic conditions and the antibodies against FGF7 can control and or modulate phosphate transport in hypophosphatemia.

### EXAMPLE 3

FGF7 protein levels were measured in the conditioned media from 2 different cell cultures explanted from the tumors and maintained in the laboratory (Ref: CA.11). Media from the cell culture that showed inhibitory activity (positive for phosphate transport activity) was compared to that of the media from the cell culture that had lost activity (negative for phosphate transport activity). FGF7 levels were measured by standard Enzyme linked immunosorbent assay well know in the art.

Results demonstrated quantitatively higher FGF7 protein levels in the conditioned media from the cultures demonstrating the inhibitory activity (1561 pg/ml) as against the levels in the conditioned media from the cultures that had lost activity (14 pg/ml).

This data further verifies that gene expression (Example 1) correlated with the increased FGF7 protein levels and the use of FGF7 polypeptide as a therapeutic for phosphate altering conditions described in the specification

### EXAMPLE 4

Sequencing of the FGF7 cDNA from the tumor samples: Normal and tumor tissues obtained from TIO patients were used to prepare cDNA preparations which was subsequently used to clone FGF-7. Multiple clones were obtained for both normal and tumor tissues and these clones were sequenced. It was observed that tumor tissues unlike the normal tissues resulted in frame shift at various regions, thus producing truncated versions of the gene. Given below are the nucleotide sequences which showed a frame shift in the sequence for the tumor tissues. The protein alignment identifies the regions that resulted in the frame shifts. Clone 410908855 resulted in the frameshift at amino acid position 178. Clone 410908832 resulted the in the frameshift at amino acid position 144.

| (SEQ ID NO: 1) | |
|---|---|
| Nucleotide sequence for the assembly 410908855 | |
| CACCAGATCTCCCACCATGCACAAATGGATACTGACATGGATCCTGCCAACTTTGCTCTACAGATCATGC | |
| TTTCACATTATCTGTCTAGTGGGTACTATATCTTTAGCTTGCAATGACATGACTCCAGAGCAAATGGCTA | |
| CAAATGTGAACTGTTCCAGCCCTGAGCGACACACAAGAAGTTATGATTACATGGAAGGAGGGGATATAAG | |
| GGGTGGAAAGTGAATTCTATCTTGCAATGAACAAGGAAGGAAAACTCTATGCAAAGAAAGAATGCAATGA | |
| CAAGAGATGAAGAATAATTACAATATCATGGAAATCAGGACAGTGGCAGTTGGAATTGTGGCAATCAAAG | |
| GGGTGGAAAGTGAATTCTATCTTGCAATGAACAAGGAAGGAAAACTCTATGCAAAGAAAGAATGCAATGA | |
| AGATTGTAACTTCAAAGAACTAATTCTGGAAAACCATTACAACACATATGCATCAGCTAAATGGACACAC | |
| AACGCAGGGGAAATGTTTGTTGCCTTAAATCAAAAGGGGATTCCTGTAAGAGGAAAAAAACGAAGAAAGA | |
| ACAAAAAACAGCCCACTTTCTTCCTATGGCAATAACTGTCGACGGC | |
| (SEQ ID NO: 2) | |
| Amino acid sequence for the assembly 410908855 | |
| MHKWILTWILPTLLYRSCFHIICLVGTISLACNDMTPEQMATNVNCSSPERHTRSYDYMEGGDIRVRRLF | |
| CRTQWYLRIDKRGKVKGTQEMKNNYNIMEIRTVAVGIVAIKGVESEFYLAMNKEGKLYAKKECNEDCNFK | |
| ELILENHYNTYASAKWTHNGGEMFVALNQKGIPVRGKKRRKNKKQPTFFLWQ | |
| (SEQ ID NO: 3) | |
| Nucleotide sequence for the assembly 410908832 | |
| GCCGTCGACAGTTATTGCCATAGGAAGAAAGTGGGCTGTTTTTTGTTCTTTCTTCGTTTTTTTTCCTCTT | |
| ACAGGAATCCCCTTTTGATTTAAGGCAACAAACATTTCCCCTCCGTTGTGTGTCCATTTAGCTGATGCAT | |
| ATGTGTTGTAATGGTTTTCAGAATTAGTTCTTTGAAGTTACAATCTTCATTGCATTCTTTCTTTGCATAG | |
| AGTTTTCCTTCCTTGTTCATTGCAAGATAGAATTCACTTTCCACCCCTTTGATTGCCACAATTCCAACTG | |
| CCACTGTCCTGATTTCCATGATATTGTAATTATTCTTCATCTCTTGGGTCCCTTTTACTTTGCCTCTTTT | |
| ATCGATCCTCAGGCACCACTGTGTTCGACAGAAGAGTCTTCTCACTCTTATATCCCCTCCTTCCATGTAA | |
| TCATAACTTCTTGTGTGTCGCTCAGGGCTGGAACAGTTCACATTTGTAGCCATTTGCTCTGGAGTCATGT | |
| CATTGCAAGCTAAAGATATAGTACCCACTAGACAGATAATGTGAAAGCATGATCTGTAGAGCAAAGTTGG | |
| CGGGATCCATGTCAGTATCCATTTGTGCATGGTGGGAGATCTGGTG | |
| (SEQ ID NO: 4) | |
| Amino acid sequence for the assembly 410908832 | |
| MHKWILTWILPTLLYRSCFHIICLVGTISLACNDMTPEQMATNVNCSSPERHTRSYDYMEGGDIRVRRLF | |
| CRTQWYLRIDKRGKVKGTQEMKNNYNIMEIRTVAVGIVAIKGVESEFYLAMNKEGKLYAKKECNEDCNFK | |
| ELILENHYNTYASAKWTHNGGEMFVALNQKGIPVRGKKRRKNKKQPTFFLWQ | |
| (SEQ ID NO: 5) | |
| Nucleotide sequence Human FGF-7 | |
| ATGCACAAATGGATACTGACATGGATCCTGCCAACTTTGCTCTACAGATCATGCTTTCACATTATCTGTC | |
| TAGTGGGTACTATATCTTTAGCTTGCAATGACATGACTCCAGAGCAAATGGCTACAAATGTGAACTGTTC | |
| CAGCCCTGAGCGACACACAAGAAGTTATGATTACATGGAAGGAGGGGATATAAGAGTGAGAAGACTCTTC | |
| TGTCGAACACAGTGGTACCTGAGGATCGATAAAAGAGGCAAAGTAAAAGGGACCCAAGAGATGAAGAATA | |
| ATTACAATATCATGGAAATCAGGACAGTGGCAGTTGGAATTGTGGCAATCAAAGGGGTGGAAAGTGAATT | |
| CTATCTTGCAATGAACAAGGAAGGAAAACTCTATGCAAAGAAAGAATGCAATGAAGATTGTAACTTCAAA | |
| GAACTAATTCTGGAAAACCATTACAACACATATGCATCAGCTAAATGGACACACAACGGAGGGGAAATGT | |
| TTGTTGCCTTAAATCAAAAGGGGATTCCTGTAAGAGGAAAAAAAACGAAGAAAGAACAAAAAACAGCCCA | |
| CTTTCTTCCTATGGCAATAACT | |
| (SEQ ID NO: 6) | |
| Amino acid sequence of FGF-7, P21781 | |
| MHKWILTWILPTLLYRSCFHIICLVGTISLACNDMTPEQMATNVNCSSPERHTRSYDYME | |
| GGDIRVRRLFCRTQWYLRIDKRGKVKGTQEMKNNYNIMEIRTVAVGIVAIKGVESEFYLA | |
| MNKEGKLYAKKECNEDCNFKELILENHYNTYASAKWTHNGGEMFVALNQKGIPVRGKKTK | |
| KEQKTAHFLPMAIT | |
| Protein Alignment Analysis (Comparison of SEQ ID NO. 2, 6 and 4) | |
| 410908855 | MHKWILTWILPTLLYRSCFHIICLVGTISLACNDMTPEQMATNVNCSSPERHTRSYDYME |
| FGF-7 | MHKWILTWILPTLLYRSCFHIICLVGTISLACNDMTPEQMATNVNCSSPERHTRSYDYME |
| 410908832 | MHKWILTWILPTLLYRSCFHIICLVGTISLACNDMTPEQMATNVNCSSPERHTRSYDYME |
| 410908855 | GGDIRVRRLFCRTQWYLRIDKRGKVKGTQEMKNNYNIMEIRTVAVGIVAIKGVESEFYLA |
| FGF-7 | GGDIRVRRLFCRTQWYLRIDKRGKVKGTQEMKNNYNIMEIRTVAVGIVAIKGVESEFYLA |
| 410908832 | GGDIRVRRLFCRTQWCLRTDKRGKVKGTQEMKNNYNIMEIRTVAVGIVAIKGVESEFYLA |
| | [symbol] |
| 410908855 | MNKEGKLYAKKECNEDCNFKELILENHYNTYASAKWTHNGGEMFVALNQKGIPVRGKKRR |
| FGF-7 | MNKEGKLYAKKECNEDCNFKELILENHYNTYASAKWTHNGGEMFVALNQKGIPVRGKKTK |
| 410908832 | MNKEGKLYAKKECNEDCNFKELILKTITTHMHQLNGHTTEGKCLLP˜˜˜˜˜˜˜˜˜˜˜˜˜˜ |
| | [symbol] |
| 410908855 | KNKKQPTFFLWQ˜˜ |
| FGF-7 | KEQKTAHFLPMAIT |
| 410908832 | ˜˜˜˜˜˜˜˜˜˜˜˜˜˜ |

Thus we have illustrated and described the preferred embodiment of our invention, it is to be understood that this invention is capable of variation and modification, and we therefore do not wish to be limited to the precis terms set forth, but desire to avail ourselves of such changes and alterations which may be made for adapting the invention to various usages and conditions. Such alterations and changes may include, for different compositions for the administration of the polypeptides according to the present invention to a mammal; different amounts of the polypeptide; different times and means of administration; different materials contained in the administration dose including, for example combinations of different peptides, or combinations of peptides with different biologically active compounds. Such changes and alterations also are intended to include modifications in the amino acid sequence of the specific polypeptides described herein in which such changes alter the sequence in a manner as not to change the functionality of the polypeptide, but as to change solubility of the peptide in the composition to be administered to the mammal, absorption of the peptide by the body, protection of the polypeptide for either shelf life or within the body until such time as the biological action of the peptide is able to bring about the desired effect, and such similar modifications. Accordingly, such changes and alterations are properly intended to be within the full range of equivalents, and therefore within the purview of the following claims. Having thus described our invention and the manner and process of making and using it in such full, clear, concise and exact terms so as to enable any person skilled in the art to which it pertains, or with which it is most nearly connected, to make and use the same.

1. A method of diagnosing a hypophosphatemic condition in a mammal, said method comprising (a) obtaining a biological sample from the mammal and (b) contacting the biological sample with a reagent which detects the presence or absence of a mutation in a nucleic acid encoding FGF7 wherein the presence of said mutation is an indication that the mammal is afflicted with the hypophosphatemic condition.

2. The method of claim 1, wherein the biological sample is selected from the group consisting of blood and urine.

3. The method of claim 1, wherein the reagent is a nucleic acid.

4. The method of claim 1, wherein the reagent is detectably labeled.

5. The method of claim 1, wherein the reagent is detectably labeled with a label selected from the group consisting of a radioisotope, a bioluminescent compound, a chemiluminescent compound, a fluorescent compound, a metal chelate, and an enzyme.

6. A method of diagnosing a hypophosphatemic condition in a mammal, said method comprising (a) obtaining a biological sample from said mammal and (b) contacting the biological sample with a reagent which detects the presence or absence of a mutant form of FGF7 polypeptide, wherein the presence of the mutant form of FGF7 polypeptide is an indication that the mammal is afflicted with the hypophosphatemic condition.

7. The method of claim 6, wherein the biological sample is selected from the group consisting of blood and urine.

8. The method of claim 6, wherein the reagent is an antibody.

9. A method of diagnosing a hypophosphatemic condition in a mammal, said method comprising (a) obtaining a biological sample from the mammal and (b) contacting the biological sample with a reagent that detects the level of FGF7 polypeptide in the sample, wherein an elevated level of FGF7 polypeptide in the sample, relative to the level of FGF7 polypeptide in a sample obtained from a control mammal, is an indication that the mammal is afflicted with said hypophosphatemic condition.

10. The method of claim 9, wherein the biological sample is selected from the group consisting of blood and urine.

11. The method of claim 9, wherein the reagent is an FGF7 antibody.

12. The method of claim 9, wherein the reagent is detectably labeled.

13. The method of claim 9, wherein the reagent is detectably labeled with a label selected from the group consisting of a radioisotope, a bioluminescent compound, a chemiluminescent compound, a fluorescent compound, a metal chelate, and an enzyme.

14. A method of diagnosing osteomalacia in a patient, said method comprising (a) obtaining a biological sample from the patient and (b) detecting the expression or lack thereof of FGF7 in the sample, wherein the expression of FGF7 is indicative of osteomalacia.

15. A method of treating a hypophosphatemic condition in a mammal, said method comprising administering to a mammal afflicted with the disorder a therapeutically effective amount of a FGF7 inhibitor selected from the group consisting of an inhibitor which reduces the level of mRNA encoding FGF7 polypeptide in the mammal, an inhibitor which reduces the level of FGF7 polypeptide in the mammal, and an inhibitor of the biological activity of FGF7 in the mammal.

16. The method of claim 15, wherein said inhibitor is selected from the group consisting of an antisense nucleic acid, a ribozyme, an antibody, a small molecule, a peptide, and a peptidomimetic.

17. A method of treating a hyperphosphatemic condition in a mammal, said method comprising administering to a mammal afflicted with the disorder a therapeutically effective amount of an isolated nucleic acid encoding FGF7.

18. The method of claim 17, wherein said isolated nucleic acid comprises a mutation that confers increased stability to the FGF7 polypeptide encoded thereby.

19. A method of treating a hyperphosphatemic condition in a mammal, said method comprising administering to a mammal afflicted with the disorder a therapeutically effective amount of an isolated FGF7 polypeptide.

20. The method of claim 19, wherein, the FGF7 polypeptide comprises a mutation that confers increased stability to said FGF7 polypeptide.

21. A method of treating a hyperphosphatemic condition in a mammal, said method comprising administering to the mammal afflicted with, a therapeutically effective amount of a reagent that increases the level of FGF7 polypeptide in said mammal.

22. The method of claim 21, wherein said reagent inhibits degradation of said FGF7 polypeptide.

23. A method of treating a hyperphosphatemic condition in a mammal, said method comprising administering to a mammal afflicted with, a therapeutically effective amount of a population of cells comprising an isolated nucleic acid encoding FGF7.

24. The method of claim 23, wherein said isolated nucleic acid comprises a mutation that confers increased stability on said FGF7 encoded thereby.

25. A method of treating a condition involving deposition of calcium and phosphate in the arteries or soft tissues of a mammal, said method comprising administering to said mammal a therapeutically effective amount of FGF7 or a reagent that increases the level of FGF7 polypeptide.
